(19) United States
(12) Patent Application Publication
(10) Pub. No.: US 2004/0152196 A1
(43) Pub. Date: Aug. 5, 2004

(54) Method for transforming kidney cells for use in a renal assist device

(76) Inventor: Paul V. Hart, Westwood, KS (US)

Correspondence Address: John C. McMahon, PO Box 30069, Kansas City, MO, 64112, US

(21) Appl. No.: 10/357,997

(22) Filed: Feb. 4, 2003

(51) Int. Cl. C12N 5/08; C12N 15/86

(52) U.S. Cl. 435/456; 435/369

(57) ABSTRACT

Human renal cells are reversibly transformed using Simian Virus 40 to produce an immortal line; the cells proliferate in vitro, serially cultured and the cultured, transformed cells are reverted to somatic phenotype. The cultured cell line serves as a reservoir of fresh cells available for reversion to a somatic phenotype by elevating the temperature. The transformed cells are grown on one face of a perfusion membrane in a renal assist device and reverted. Patient blood is circulated through the device for contacting the reverse face of the perfusion membrane. Molecules in the blood pass through the membrane and into contact with the renal tubule cells. The reverted cells perform various cellular functions on the molecules in the blood, such as waste product removal and metabolic and endocrine functions.

### BACKGROUND OF THE INVENTION

The present invention is broadly concerned with an improved kidney assist device. More particularly, it is concerned with a method for reversibly transforming a line of renal cells to permit serial subculture, subsequently reverting the cells to a somatic phenotype, followed by use of the cells in a device for perfusion with the blood of a patient.

The kidneys serve to remove waste products from the blood and to perform various metabolic and endocrine functions, such as ammonia neogenesis and excretion, glutathione reclamation, vitamin D3 activation and cytokine homeostasis. When the kidneys fail as a result of End Stage Renal Disease (ESRD) or acute renal failure (ARF), these functional abilities are compromised. Hemodialysis is the therapy of choice for ESRD and ARF patients. Hemodialysis replaces the normal kidney glomerular function of small solute clearance with mechanical filtration of the blood. Conventional hemodialyzers operate by pumping blood from a patient through a chamber equipped with a plurality of capillary hollow fibers. The fibers are constructed from a semipermeable membrane material having a preselected exclusion limit that determines the size of the molecules that may pass through the membrane. A countercurrent of a dialysis fluid or dialysate is circulated around the outside of the fibers. Small solute waste products in the blood such as urea and creatinine diffuse through the fibers and into the dialysate, and the purified blood is returned to the patient.

The adequacy of such dialysis treatment is generally monitored by measuring urea kinetics as Kt/V, or dialyzer clearance multiplied by time divided by body water volume. Kt/V provides a measurement of the amount of urea along with excess fluid removed from the body during treatment (the “small solute clearance level”). Urea is used as a benchmark because it is thought to be indicative of the levels of other waste products in the blood. An alternate Hemodialysis Product (HDP) is obtained from the product of the number of hours per dialysis session times the square of the number of dialysis sessions per week, and is thought to reflect increased clearance levels of larger molecules.

The incidence of End Stage Renal Disease (ESRD) is increasing throughout the world and mortality rates among U.S. dialysis patients currently exceed 20%. Various studies have been undertaken to in order to determine whether the type of dialysis membrane or the dialysis dose may be adjusted in order to reduce hemodialysis mortality. Studies based on the Kt/V measurement suggest that longer dialysis sessions (and increased small solute clearance levels) will not enhance patient survival. Studies based on the HDP measurement suggest that more frequent, short dialysis sessions (and increased middle molecule clearance levels) will decrease patient mortality.

However, these studies and the underlying Kt/V and HDP formulae address only the glomerular solute clearance or waste removal function of the kidneys. Neither current dialysis therapy nor its associated investigational tools addresses the metabolic and endocrine functions performed by the renal tubule cells, yet many researchers in the field attribute high patient mortality rates to lack of ammonia metabolism, glutathione reclamation, vitamin D3 activation, cytokine homeostasis and other cellular metabolic functions and factors provided by renal tubule cells.

There have been attempts to use renal tubule cells as an adjunct to conventional hemofiltration devices by constructing cartridges that incorporate xenotransplanted animal and human cells. However, such renal tubule assist devices (RADs) have proved to be of only limited utility, since primary renal tubule cells have a finite life span and eventually experience senescence. Thus, RADs incorporating primary renal cells can provide relief from symptoms of chronic renal failure in only a small population for nonextended periods of time. And because once the cells become senescent, they lack the desired somatic functionality, a consistent supply of fresh kidney tissue would be required to construct new RAD units and to maintain cell vitality in RADs in clinical use.

Various attempts have been made to establish a supply of donor kidneys for such use. While many animals are suitable for breeding to maintain such a supply, pigs are considered to be most suitable because of the similarities between porcine and human cells. However, endogenous retroviruses that are incorporated into the porcine genome (PERVs) have been reported to be capable of expressing virions capable of infecting human cells in coculture. At the present time, there is no known means of removal of such retroviruses and they present a risk of transmission to immunosuppressed human recipients. This potential risk of pig virus transmission to human tissue has caused the Food and Drug Administration to closely monitor research clinical trials using porcine cells for this purpose and makes it unlikely that porcine cells will be approved for use in kidney assist devices in the near future.

Live human kidney cells are clearly most suitable for usage in RADs and human cells are available in kidney transplant discards. However, cells used in each RAD must be histocompatible with the cells of the dialysis recipient and the need for such tissue far exceeds the quantity available. Only a tissue culture method appears likely to be capable of providing the cell volume necessary to produce and maintain RADs for widespread use in the treatment of ESRD. Attempts to employ stem cells to establish such a human renal tubule cell supply have not been successful. Conventional methods for preparing permanent human cell lines by serial subculture yield sufficient cell quantities, but the cell lines lack somatic function and are unable to remove waste products and to perform the metabolic and endocrine functions necessary in a RAD.

Accordingly, there is a need for a method for serially culturing human renal cells while retaining their abilities to remove waste products and to perform metabolic and endocrinologic somatic functions.

Detailed Description of the Invention

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

A method for transforming kidney cells in accordance with the invention includes the steps of obtaining a quantity of live kidney cells, preparing a primary cell culture, transforming the cells, serially subculturing the cells and inducing the cells to revert to the somatic phenotype.

The primary cell culture may be derived from a tissue explant, such as a harvested but unmatched human kidney, from a biopsy, such as an open biopsy at surgery or a closed biopsy at diagnosis, from a living related donor, or from a donated cadaver kidney. The culture may also be obtained from an established transformed cell line. The preferred cells are obtained from the nephrons, particularly renal tubule cells, with proximal tubule cells being particularly preferred because they are capable of a broad range of somatic functions, including metabolic and endocrine functions. However it is foreseen that other nephron cell types such as the myoepithelial cells of the afferent arteriole cells and cells of other components of the juxtaglomerular apparatus as well as other cells of the kidney may also be employed, depending on the desired somatic functions.

A primary cell culture is established using known media, atmosphere and procedures. In general, the source tissue sample is dissociated either physically or by enzymatic digestion, such as for example using trypsinase, to yield dispersed primary cells suitable for seeding a culture medium and subsequent cell growth. Quantities of both primary cells and stock primary cell cultures may also be stored frozen, for example, in liquid nitrogen, preferably following addition of a cryoprotective agent such as glycerol or DMSO.

The renal tubule cells are preferably transformed by infection such as transfection or lipofection using a temperature sensitive viral transforming agent or promoter using known materials and methods. While either a DNA or RNA-containing virus may be employed, a DNA-containing virus that will not continue to produce virions such as a papovavirus is preferred, such as an amino acid substitution mutant of Simian Virus 40 large tumor antigen (SV40 tsA mutant). Temperature sensitive SV40 mutants such as tsA 28, (or tsA 393W-C); tsA 30 (or tsA 357R-K), tsA 255 (or tsA 422W-C), tsA 209 (or tsA 427P-L) and tsA 58 (or tsA 438A-V) are particularly preferred transformational agents. Primary renal tubule cells that have been treated with a transforming agent may be characterized as proliferating, exhibiting increased capacity to persist in serial subcultures, also exhibiting increased rate of growth in vitro and loss of contact inhibition. Those skilled in the art will appreciate that any temperature sensitive SV40 mutant or other transforming agent capable of inducing these properties in human renal tubule cells may also be employed.

The transformed renal tubule cells form clones or plaques that are next serially subcultured in order to select stably transformed cells from transiently transformed or so-called abortive cells. The stably transformed cells are then harvested and transferred to fresh growth medium for establishing single or multiple serial subcultures of continuous cell lines. It is foreseen that a supply or reservoir of subcultured transformed cells may be conveniently maintained in a bioreactor.

The proliferating, transformed cells are next cultured onto the exterior surfaces of capillary tubes formed of a biocompatible material in a known renal assist device using known methods. The exterior surfaces of the capillary fibers are coated with a matrix such as bovine collagen type IV (Becton Dickinson, Bedford, Mass.). Broadly speaking, a renal assist device includes a capillary hollow fiber cartridge of conventional construction. One such device is the F-40 hollow fiber dialyzer produced by Fresenius. The transformed cells are seeded onto the outer surfaces of the fibers and preferably grown to a confluent monolayer. If the cell coated RAD cartridges are perfused with a nutrient-containing solution in order to maintain cell adhesion, they may be stored until usage.

In use, a RAD cartridge containing hollow fiber capillary tube bundles, coated with transformed cells is placed in a water bath and elevated to a temperature preselected to permit reversion of the transformed renal tubule cells to a somatic state, or a temperature of from about 36° C. to about 41° C., preferably of about 39° C. Alternatively, the RAD cartridge may be warmed by circulation of prewarmed dialysate. The warmed RAD cartridge is coupled in series after a standard hemodialysis unit. Blood from a patient to be treated is permitted to flow from a selected artery and is passed through a conventional hemodialysis machine, such as the DC30 produced by Amicon Corporation, Danvers, Mass. Following conventional dialysis, the blood is routed through the capillary tubes in the RAD cartridge while a dialysis solution is circulated over the external surfaces of the tubes in countercurrent relationship. Molecules in the blood flowing through a capillary lumen pass through the capillary membrane to contact temperature-shifted renal tubule cells. The renal tubule cells perform various metabolic and endocrine functions on the molecules in the blood, such as, for example, ammonia neogenesis and excretion, glutathione reclamation, 1,2,5 dihydroxy vitamin D₂ production and cytokine homeostasis. The treated blood is then cooled to about 37.5° C. and is routed for venous return to the patient.

It is also foreseen that the RAD may be mounted downstream of a conventional hemofiltration device and the transformed cells may be seeded onto the luminal surfaces of the capillary fibers for growth to a confluent monolayer. In such a method, the patient's blood is first passed through a conventional high flux hemofilter. After an initial filtration period of about 1 hour, the heated RAD cartridge is added to the circuit to receive the output of the hemofilter. The ultrafiltrate is routed into the RAD capillary tubes or luminal compartment into contact with the transformed cells, while the postfiltered blood is routed into the shell or extracapillary space separating the capillary tubes. The ultrafiltrate is monitored and electrolyte replacement solution is infused as necessary. The effluent blood from the RAD cartridge is cooled to body temperature and routed for venous return to the patient.

In this manner, not only are toxic small solutes removed from the blood by conventional hemodialysis, but dissolved molecular species are taken up for metabolism and endocrinologic products are generated by the renal tubule cells to more closely approximate normal physiological functions of the kidneys in patients. It is also foreseen that the circuit may include one or more additional cartridges, as, for example, an adsorbent cartridge.

### EXAMPLE I

In this example, human renal proximal tubule cells (PTCs) are transformed into proliferating cells using the SV40 Variant tsA 28.

Human kidney cortical tissue is obtained from a tissue explant, biopsy, live donor, or cadaver. Primary PTC cultures are prepared generally in accordance with the method described by Humes et al., Metabolic Replacement of Kidney Function in Uremic Animals With a Bioartificial Kidney Containing Human Cells, American Journal of Kidney Diseases, Vol. 39, No. 5, 2002, Pp 1078-1087.

Materials

Primary Culture Incubation Solution:

1.14 mM Calcium chloride and

0.73 mM Magnesium Sulfate

in Dulbecco's Modified Eagle Medium (DMEM)

(BioWhittaker, Walkersville, Md.)

Primary Culture DNase Solution:

5.5 mg/ml DNase (Sigma, St. Louis, Mo.) in

1.14 mM calcium chloride and

0.73 mM magnesium sulfate in

DMEM

Primary Culture Collagenase Solution:

10 mg/ml collagenase, Class IV (Worthington, Freehold, N.J.) in

1.14 mM calcium chloride and

0.73 mM magnesium sulfate

in DMEM and

1.5 ml 4° C. DNase solution

Primary Culture Growth Medium:

UltraMDCK Media No. 12-749Q (BioWhittaker,

Walkersville, Md.) supplemented with:

1 m/L insulin, transferrin, ethanolamine, selium (BioWhittaker No. 17-839Z)

60 μg/L epidermal growth factor No. 236-EG (R&D Systems, Minneapolis, Minn.)

0.010 ml/L Triiodothyronine (BioWhitaker No. CC-4211)

Serum-Free Medium:

Williams' E medium (BioWhittaker) supplemented with:

5% fetal calf serum (FCS)

2.1 μM prednisolone

0.038 μM glucagon

0.16 U/ml insulin

200 U/ml penicillin

137 μM streptomycin

Lipofection Solutions:

Solution A: 16 μg DNA in 800 μl serum-free medium

Solution B: 120 μl Lipofection (Invitrogen Corp.; Carlsbad, Calif.) plus 680 μl serum-free medium

HBS Buffer

8.18 g/l NaCl

5.95 g/l HEPES

0.2 g/l Na₂HPO₄

adjust pH to 7.05

Methods

Preparation of Primary Culture

The cortex is minced and tissue samples are dissected to isolate one or more regions containing primarily renal tubule cells, and the isolated tissue is sliced into fragments of approximately 1 cubic mm size.

Multiple flasks are prepared, each containing 3.5 ml of 4° C. collagenase solution added to 20 ml of 37° C. prewarmed sterile incubation solution. The flasks are placed in a beaker containing prewarmed 37° C. water. Up to 25 g of minced tissue is added to each flask, which is then placed in a 37° C. carbon dioxide incubator for 15 minutes to permit tissue digestion. The flasks are then removed and 25 ml of 4° C. DMEM is added to each flask to stop the reaction. The tissue solutions are then strained through an 850 μm sieve. Each tissue filtrate is resuspended in new solutions as previously described, the procedure being repeated up to six times. As the quantity of undigested tissue filtrate is reduced, filtrates from the flasks are combined.

The digested tissue is centrifuged at 53 g for 15 minutes, and the pellets resuspended in 4° C. DMEM and strained using a 710-μm sieve. The tissue is resuspended and centrifuged as before, and strained using a 600-μm sieve. The filtrate is suspended in growth medium and transferred to plates.

The cells are incubated at 37° C. in a 5% carbon dioxide atmosphere, with medium changed at 1-3 day intervals. Confluent cells are passaged at a 1:2 ratio. Growth medium containing 0.033 mg/l retinoic acid (Sigma, St. Louis, Mo.) is added via medium exchange 24 hours before the initial passage, and this medium is used for all subsequent media exchanges. The cells are passaged every 3-7 days thereafter.

After several passages, the cells are seeded onto 60 mm collagen-coated petri dishes. The next day the cells are transfected using Liebowitz L-15tsA 28 SV40 virus (10⁶-10⁷ virus particles per cell)

Mutant Cloning

Bacterial cells are infected with the SV40 mutant tsA 28 to obtain an available reservoir of tsA 28 in the following manner:

The tsA 28 mutant is obtained (from Robert Haltiwanger, Ph.D., State University of New York, Stony brook) in blue script plasmid form on filter paper and is eluted with ethanol. 10 ng DNA is next added to 50 μl competent E. coli XL 10 Gold bacteria (Stratagene, USA), which are placed on ice for 5 minutes, then moved to a 42° C. water bath for 1 minute, returned to ice for 2 minutes; and spread on 60 mm Agar-amp-dishes at a concentration of 30 μl per dish. The cultures are incubated overnight at 37° C.

Hirt-Extraction

The viral DNA is next extracted from the host E. coli cells using the method of Hirt as follows:

Growth medium is removed from the infected cells. 1.0 ml of 0.6% SDS in 0.01M EDTA (pH 7.5) is added and the cells incubated for 10-20 minutes at ambient temperature. The lysate is transferred to a centrifuge tube; 5 M NaCl is added to yield a 1M concentration, the tube is gently mixed and incubated at 4° C. for at least 8 hours. The tube is next centrifuged for 30 minutes at 17000 g at 4° C. The viral DNA may be recovered in the supernatant and the two fragments (5242 bp and 2964 bp) confirmed by gel electrophoresis.

Transfection of PTC Cells with TSA 28

A DNA solution is prepared by solving 2.5 μg tsA 28 DNA in TE-buffer (pH 7.4, at least 0.1 μg/μl) and adding serum-free medium to make up a total volume of 150 μl. 15 μl of SuperFect Transfection Reagent (Qiagen GMBH; Hilden, Germany) is added to the DNA solution and vortexed for 10 seconds. The samples are incubated for 5-10 minutes at ambient temperature to permit complex formation. The medium is gently aspirated from the cells and they are washed once with phosphate buffered saline (PBS). I ml of transfection medium is added to the reaction tube containing the transfection complexes and the tube is vortexed for about 10 seconds. The total volume is immediately transferred to the PTC cultures in the 60 mm dishes. The PTC cultures are incubated with the complexes for 2-3 hours, followed by removal of the medium, washing with PBS and addition of new serum-free medium. Transfection is verified with an immunofluorescense staining of the large T-antigen. Plaques of stably transfected cells are selected and serially cultured as immortalized cell lines. The medium for the immortalized cell line is changed at 3-4 day intervals.

### EXAMPLE II

In this example, human renal proximal tubule cells (PTCs) are transformed into proliferating cells by lipofection using the SV40 Variant tsA 28.

PTC cells from the primary culture are incubated on 100 mm collagen coated petri dishes until they are 40-60% confluent. DNA solution is prepared as described in Example I and is used to prepare Lipofection Solution A. Lipofection Solution B is prepared and allowed to stand at ambient temperature for 30-40 minutes. Solution A and Solution B are combined, mixed gently and incubated at ambient temperature for 10-15 minutes to permit complex formation. The cells in the dishes are washed 2-3 times with serum-free medium. For each 100 mm dish 6.4 ml of serum-free medium is added to the tube containing the lipofectin-DNA complex and mixed gently. The total volume of the complex solution is immediately transferred to the PTC cultures in the 100 mm dishes. The PTC cultures are incubated with the complexes for 24 hours, followed by washing with PBS and addition of new serum-free medium. Lipofection is verified as in Example I. Plaques of stably transfected cells are selected and serially cultured as immortalized cell lines.

### EXAMPLE III

In this example, PTCs are transformed into proliferating cells by calcium phosphate coprecipitation using the SV40 Variant tsA 28.

PTC cells from the primary culture are incubated on 60 mm collagen coated petri dishes until they are 40-60% confluent. DNA solution is prepared by solving 20 μg tsA 28 DNA in 750 μl HBS buffer and mixing gently. While vortexing, 48 μl of 2M calcium chloride is added. The DNA-calcium phosphate complexes are incubated 20 minutes at ambient temperature. The transfection reagent is spread evenly on the culture dish and the cells are incubated overnight at 37° C. The cells are washed twice with PBS and new medium is added. Transfection is verified as in Example I. Plaques of stably transfected cells are selected and serially cultured as immortalized cell lines.

### EXAMPLE IV

In this example, PTCs are transformed into proliferating cells by infection with the SV40 Variant tsA 255. This mutant is obtained (from Janice Chou, Ph.D., National Institutes of Health, Bethesda, Md.) in the form of viral particles in solution. PTC cells from the primary culture are seeded onto 100 mm collagen coated dishes and incubated for 24 hours. A second collagen layer is added and the cells are infected by addition of 1 ml of the virus suspension in each dish and the cells are incubated overnight at 37° C. The cells are washed twice with PBS and new medium is added. Transfection is verified as in Example I. Plaques of stably transfected cells are selected and serially cultured as immortalized cell lines.

It is to be understood that while certain forms of the present invention have been illustrated and described herein, it is not to be limited to the specific forms or arrangement of parts described and shown.

What is claimed and desired to be secured by Letters Patent is as follows:

1. A method for reversibly transforming and subculturing renal tubule cells for reversion to a somatic phenotype and comprising:
a) providing a quantity of renal tubule cells;
b) reversibly transforming said kidney cells to cause the cells to proliferate in vitro;
c) serially culturing said reversibly transformed cells to maintain a supply of cells; and
d) inducing said cultured, reversibly transformed renal tubule cells to revert to the somatic phenotype thereof.

2. The method as set forth in claim 1, wherein said renal tubule cells are human cells.

3. The method as set forth in claim 1, wherein said cells are reversibly transformed by a temperature sensitive Simian Virus 40 mutant.

4. The method as set forth in claim 3, wherein said temperature sensitive Simian Virus 40 mutant is selected from the group consisting of tsA 28, tsA 30, tsA 255, tsA 209, and tsA 58.

5. The method as set forth in claim 1, wherein:
a) said cultured, reversibly transformed kidney cells are temperature sensitive; and
b) step (d) further includes the step of elevating the temperature of said reversibly transformed renal tubule cells to a temperature of at least about 39° C.

6. The method as set forth in claim 1, in combination with a method of hemodialysis including the step of:
a) using said cells in a renal assist device.

7. The method as set forth in claim 1, including the steps of:
a) providing a renal assist device having a semipermeable membrane with a first face and an opposed second face;
b) placing said reversibly transformed renal tubule cells on said perfusion membrane first face;
c) subsequently causing said cultured, reversibly transformed renal tubule cells to revert to the somatic phenotype thereof;
d) withdrawing blood from the body of an organism;
e) subjecting said blood to a hemodialysis process; and
f) contacting said membrane second face with said withdrawn blood and permitting molecules in the blood to pass through the membrane to contact said renal tubule cells for permitting said cells to perform cellular functions on said molecules blood.

8. The method as set forth in claim 1, wherein:
a) said cultured, reversibly transformed kidney cells are temperature sensitive; and
b) step (c) further includes the step of elevating the temperature of said reversibly transformed renal tubule cells to a temperature of at least about 39° C.

9. The method as set forth in claim 1, including the step of:
a) culturing said reversibly transformed renal tubule cells in a bioreactor.

10. A method of processing blood and comprising the steps of:
a) providing a renal assist device having a semipermeable membrane with a first face and an opposed second face;
b) providing a quantity of renal tubule cells reversibly transformed to proliferate in vitro;
c) culturing a quantity of said reversibly transformed renal tubule cells on said perfusion membrane first face;
d) causing said cultured, reversibly transformed renal tubule cells to revert to the somatic phenotype thereof;
e) withdrawing blood from the body of an organism; and
f) contacting said membrane second face with said withdrawn blood and permitting selected molecules in the blood to pass through the membrane to contact said renal tubule cells for permitting said cells to perform cellular functions on said molecules in the blood.
